(19) United States
(12) Patent Application Publication
(10) Pub. No.: US 2005/0267373 A1
(43) Pub. Date: Dec. 1, 2005

(54) Tool insertion guidance device for use with a medical imaging system

(76) Inventor: Doohi Lee, Plano, TX (US)

Correspondence Address: Doohi Lee, 5508 Ash Creek Lane, Plano, TX, 75093, US

(21) Appl. No.: 10/856,608

(22) Filed: May 28, 2004

(51) Int. Cl. A61B 8/12

(52) U.S. Cl. 600/471; 600/437

(57) ABSTRACT

A tool insertion guidance device is disclosed for guiding an insertion of a surgical tool to a target area monitored by a medical imaging tool. The device comprises a protractor assembly attached to the medical imaging tool having a predetermined moving track, a hollow guide tube held by the protractor assembly for receiving the surgical tool, the hollow guide tube assuring an insertion of the surgical tool to be within an imaging plane of the medical imaging tool, and an adjusting means for moving a first end of the hollow guide tube along the moving track for defining an insertion angle of the surgical tool.

### BACKGROUND

The present disclosure relates generally to medical devices, and more particularly to a variable-angle needling device that may be used in a variety of surgical procedures.

Ultrasound is one of the simplest, safest, and most versatile imaging technologies available. Handheld ultrasound transducers, which provide ultrasound imaging at a relatively low cost, may be maneuvered quickly and effectively on the skin of a patient by a skilled technician. The transducer first emits ultrasonic sound waves into the body of a patient, and then receives reflections of ultrasonic sound from physiological targets. An ultrasound image is then constructed through hard reflections or interfaces between regions in which sound travels at different velocities. A remote screen displays an image that is interpreted from the data produced by ultrasonic sound waves reflecting from somatic structures. An image is produced of a cross-section of the body of a patient, thereby providing a view that is impossible without invasive procedures. For example, such an image may be extremely helpful in accurately delivering medication or targeting biopsy tool to a target treatment area.

In medical science, invasive sampling or treatment of a discovered target is of paramount importance. However, projecting a needle or other biopsy instrument accurately towards such a target has historically been difficult. The size of the apparatus, in some cases, has been large and damaging. Typically, there has been inadequate or no angular reference. If the target is missed, the angle of penetration cannot be changed without a full withdrawal. This means that more than one penetration, with the attendant additional trauma, may have been necessary to reach the target tissue.

Therefore, desirable in the art of medical devices is a device that provides accurate angular prediction for needle penetration, for secure support of the needle in a correct position to approach the target tissue, and for secure support of the needle such that the needle remains in the plane of the ultrasonic scan during its entire travel.

### SUMMARY

In view of the foregoing, this disclosure provides examples of a variable-angle tool insertion guidance device. This guidance device provides an accurate angular prediction for the penetration of a surgical tool.

Three examples of a tool insertion guidance device are disclosed for guiding an insertion of a surgical tool to a target area monitored by a medical imaging tool of a medical imaging system. The device comprises a protractor assembly attached to the medical imaging tool having a predetermined moving track, a hollow guide tube held by the protractor assembly for receiving the surgical tool, the hollow guide tube assuring an insertion of the surgical tool to be within an imaging plane of the medical imaging tool, and an adjusting means for moving a first end of the hollow guide tube along the moving track for defining an insertion angle of the surgical tool. One engineering solution is also disclosed to allow the medical imaging tool to rotate about an axis.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIGS. lA-lC illustrate a variable-angle needling device with securing pins in accordance with a first example of the present disclosure.

FIGS. 2A-2C illustrate a variable-angle needling device with “virtual” axis in accordance with a second example of the present disclosure.

FIGS. 3A-3B illustrate a variable-angle needling device that allows the medical imaging tool to rotate about an axis in accordance with a third example of the present disclosure.

### DESCRIPTION

This disclosure will provide a detailed description of a variable-angle surgical tool insertion guidance device, which may provide an accurate and stable mechanism with which to target a hypodermic treatment area while using a medical imaging system. The examples below use a needling device as the illustrated examples, but it is understood that tools other than the needling device can be guided in the same manner. Further, ultrasonic tools are used for illustration below, but the disclosed device can be used with any radiology or medical imaging system.

FIG. 1A illustrates a variable-angle needling device 100 in accordance with a first example of the present disclosure. The variable-angle needling device 100 may be used for guiding a hypodermic needle to a somatic target that is discovered through and remotely displayed by an ultrasonic diagnostic machine. It is however understood by those skilled in the art that any reference to a hypodermic needle may be replaced by other surgical tools without deviating from the spirit of this disclosure.

An ultrasonic transducer 102 emits an angular sweeping beam with a planar, pie-cut-shaped field of view. The beam widens downward from the center of the bottom of the ultrasonic transducer 102, and sweeps in the plane as represented by FIG. 1A. It is however understood that other ultrasonic transducers that produce other geometries of ultrasonic beams may be used. The transducer also receives ultrasonic echoes and sends the information to a remote processor (not shown), where the data are analyzed and the ultrasound image displayed. Two protractor-like plates of a protractor assembly 104 force a hollow guide tube 106 to be in the plane of the ultrasonic beam. The hollow guide tube 106 includes one or two attached securing pins 108 at its lower end. The attached securing pins 108 define an axis for the rotation or an entry point of the hollow guide tube 106 in the imaging plane of the ultrasonic beam. As it is understood that the “plane” in which the ultrasonic beam captures an image is not “paper thin,” it is really a small volume with predetermined dimensions. Since the image is largely two-dimensional, the term “plane” is used for convenience. The hollow guide tube 106 also includes one or two attached guide pins 110 at its upper end. The two attached guide pins 110 act as followers within a moving track, such as an arced slot 112, in the two protractor-like plates of the protractor assembly 104. It is understood that the arced slot 112 may not have a “smooth” track as it may have multiple “teeth” between which the guide tube will be stabilized. As the guide tube only provides a practitioner an easy means for inserting the needle instead of relying on observation judgment alone, the guide tube may not need to be “locked” rigidly because the practitioner is still in full control of the needle. As such, the teeth pattern track may be sufficient to hold the guide tube without any other locking mechanism.

The device 100 may also have a position-locking mechanism. The mechanism may include a locking nut 114 which screws onto one of the guide pins 110. The position of the hollow guide tube 106 is locked at a selected angle, which may be indicated on the face of the protractor assembly 104. When the locking nut 114 is released, the hollow guide tube 106 may be adjusted to a newly selected angle, as indicated at a position 116, where it can again be locked.

Depending on the length of the hollow guide tube 106, it may be “pulled in or out” with an available range as long as a portion of it is secured between the pins 108 and 110. This allows the flexibility to receive a hypodermic needle 118 most appropriate for a particular treatment. The hypodermic needle 118 may include an adapter 120, which facilitates the attachment of a syringe, not shown. The protractor assembly 104 may be attached to the transducer 102 by a bracket 122. The bracket 122 holds the protractor assembly 104 in a fixed position relative to the transducer 102 by conveniently adjusting the lengths of the belts or straps 124 and 126 around different types and sizes of ultrasonic transducers. It is understood that various securing means can be used here for arranging the protractor assembly and the transducer so that they are aligned in such a way that the needle inserted will always be within the display view of the ultrasonic transducer. It is further understood that the best and easiest way to achieve this result is to assure that the inserted needle travels in the same plane as the ultrasonic transducer beam.

In operation, the base 128 of the device 100 is placed on the skin of a patient, so that the ultrasonic beam projects vertically downward into the tissue of the patient. The display screen of the ultrasonic diagnostic machine is calibrated to show the depth of a discovered somatic target. When the transducer is moved across the skin of the patient, the target tissue may be displayed on a properly marked region of the screen. The depth of the target tissue is measured. By means of a table of values or computer software, an appropriate angle is then selected for the hollow guide tube 106 so that the hypodermic needle 118 will intersect the target tissue at the measured depth. The hollow guide tube 106 is locked at the selected angle. As the hypodermic needle 118 is inserted into the hollow guide tube 106, the needle is automatically pointed toward the target tissue. As the needle is further inserted through the skin and into the tissue of the patient, the progress of the needle toward the target tissue becomes visible on the screen. The device 100 maintains the coplanarity of the ultrasonic beam and the hypodermic needle 118 at all times. The arrival of the tip of the hypodermic needle 118 at the target tissue may be monitored on the screen and stopped when appropriate. Further, the injection of material into, or withdrawal of material from, the target tissue can also be monitored on the screen. At the end of the treatment, the hypodermic needle 118 is withdrawn through the hollow guide tube 106, which may be locked in position for smooth withdrawal.

It is understood that the hollow guide tube 106 may be removable and disposable, and that the device 100 may be easily cleaned. This disposable feature increases the sanitary safety of the device 100.

FIG. 1B illustrates an isometric view 130 of the device 100 for guiding the hypodermic needle 118 to a somatic target that is discovered and displayed by an ultrasonic diagnostic machine. The view 130 shows how the protractor assembly 104 may be attached to the ultrasonic transducer 102 via the bracket 122. The protractor assembly 104 may open by means of a hinge or other mechanism, not shown, to allow the removal of the disposable guide tube. As the hypodermic needle 118 is withdrawn from the patient, blood may contaminate the hollow guide tube 106. Therefore, a fresh and clean guide tube 106 may be inserted into the protractor assembly 104 for the next patient.

FIG. 1C illustrates a detailed view 132 of a guiding mechanism. Two attached securing pins 108 are used to act as pivots for the hollow guide tube 106. The hypodermic needle 118 can be inserted into the hollow guide tube 106 and then into the patient tissue. A locking mechanism is also presented, whereupon the locking nut 114 is screwed onto one of the guide pins 110.

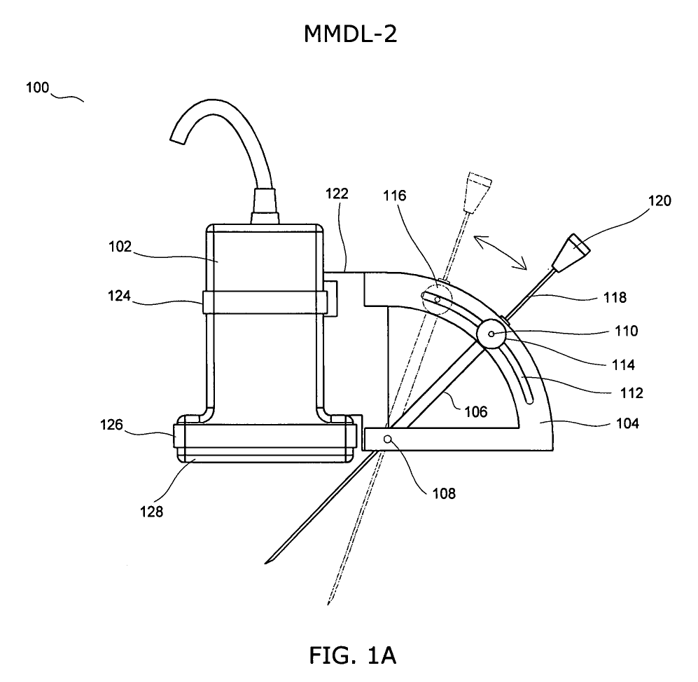

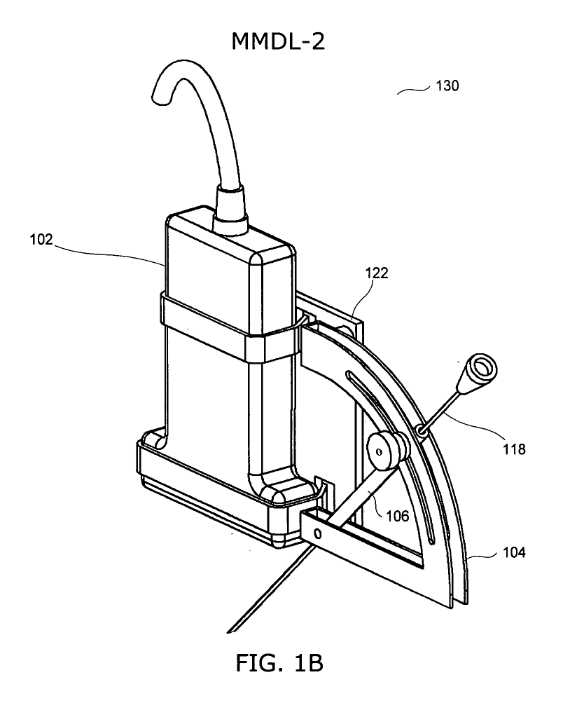

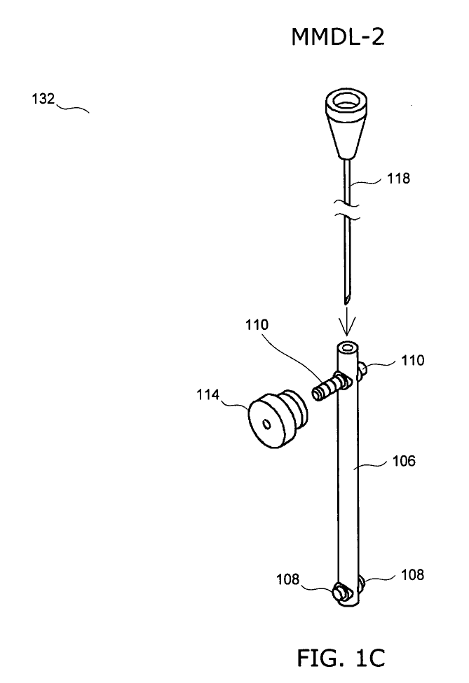

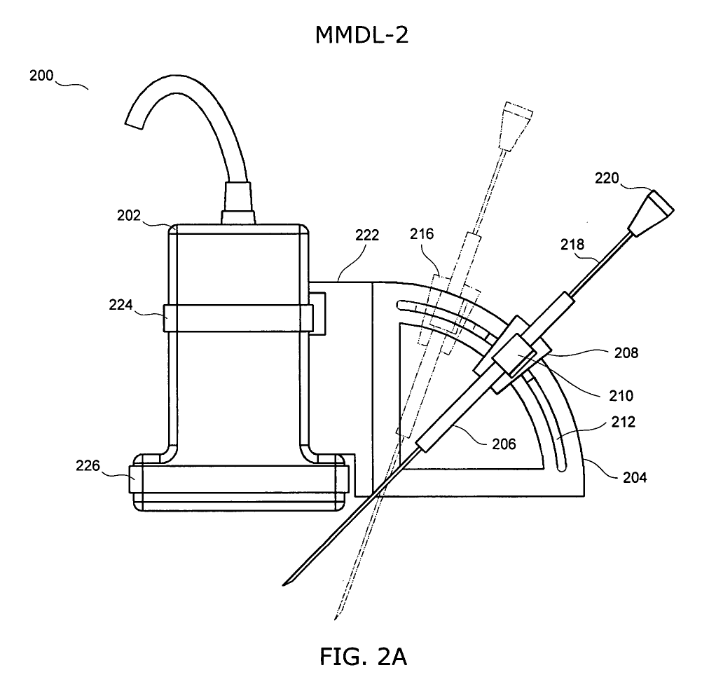

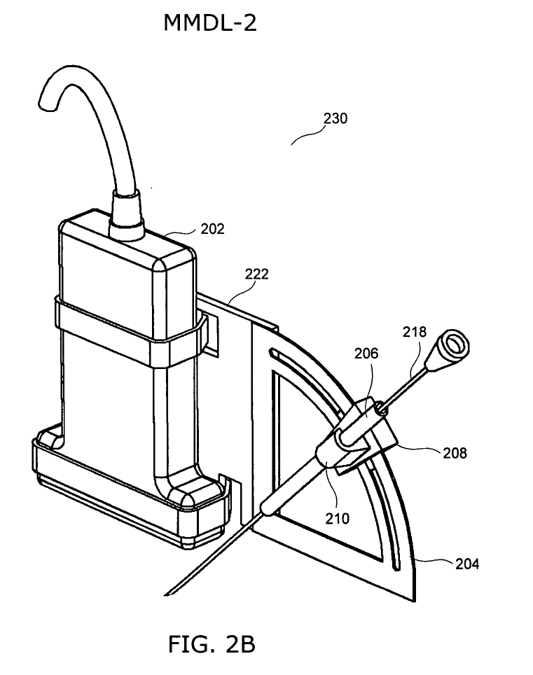

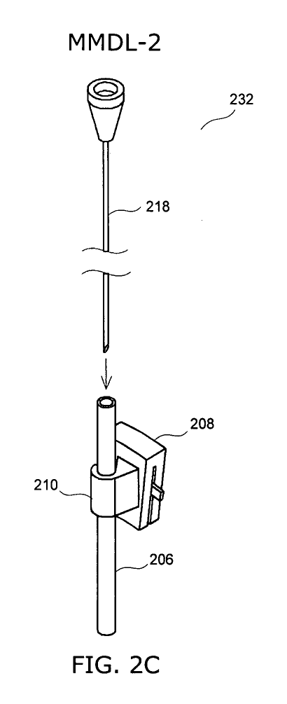

FIG. 2A illustrates a variable-angle needling device 200 in accordance with a second example of the present disclosure. An ultrasonic transducer 202 emits an angular sweeping beam with a planar, pie-cut-shaped field of view. The beam widens downward from the center of the bottom of the transducer 202, and sweeps in the plane of the drawing. It is however understood that there are other ultrasonic transducers that produce ultrasonic beams of different geometries. The transducer 202 also receives ultrasonic echoes and sends the information to the device 200, where it is displayed as an image on a screen of the ultrasonic diagnostic machine. A protractor assembly 204 (only one protractor-like plate, as opposed to two plates in the protractor assembly 104) is parallel to the plane of the ultrasonic beam. A hollow guide tube 206 is held in alignment by a bracket 208. The bracket 208 has an attached snap-in clamp 210, which holds the hollow guide tube 206 in the plane of the ultrasonic beam. The hollow guide tube 206 snaps into the clamp 210 and is therefore always assured to be in the plane of the ultrasonic beam. In this example, the needle inserted is always through a “virtual” point roughly around the lower left corner of the protractor assembly. The bracket 208 acts as a follower within an arced slot 212 in the protractor assembly 204 without changing its orientation so that the virtual point mentioned above is maintained no matter which final position the bracket 208 is locked on the slot 212.

The device 200 may also include a position-locking mechanism. The mechanism may be enabled by a locking nut, not shown, screwed onto a threaded pin, not shown. The position of the hollow guide tube 206 is locked at a selected angle, which is indicated on the face of the protractor assembly 204. When the locking nut is released, the hollow guide tube 206 may be adjusted to a newly selected angle as indicated at a position 216, where it can again be locked. As long as the hollow guide tube 206 is secured to the bracket 208, it can be flexible as long as it is within an available range so that it can accommodate a hypodermic needle 218 in the most appropriate manner. The hypodermic needle 218 may include an adapter 220, which facilitates the attachment of a syringe, not shown. The protractor assembly 204 is attached to the ultrasonic transducer 202 by a bracket 222. The bracket 222 holds the protractor assembly 204 in a fixed position relative to the ultrasonic transducer 202 by conveniently adjusting the lengths of the belts or straps 224 and 226 around different types and sizes of ultrasonic transducers.

FIG. 2B illustrates an isometric view 230 of an apparatus for guiding the hypodermic needle 218 to a somatic target that is discovered and displayed by an ultrasonic diagnostic machine. This view shows how the protractor assembly 204 may be attached to the ultrasonic transducer 202 via the bracket 222. As the hypodermic needle 218 is withdrawn from the patient, blood may contaminate the hollow guide tube 206. Therefore, a fresh, clean guide tube 206 may be inserted into the clamp 210 of the bracket 208 of the protractor assembly 204 for the next patient.

FIG. 2C illustrates a detailed view 232 of a guiding mechanism. The hollow guide tube 206 is held in alignment by the bracket 208. The bracket 208 includes the attached snap-in clamp 210. After the hollow guide tube 206 is snapped into the clamp 210, the hypodermic needle 218 may be inserted into the hollow guide tube 206 and then into the patient tissue.

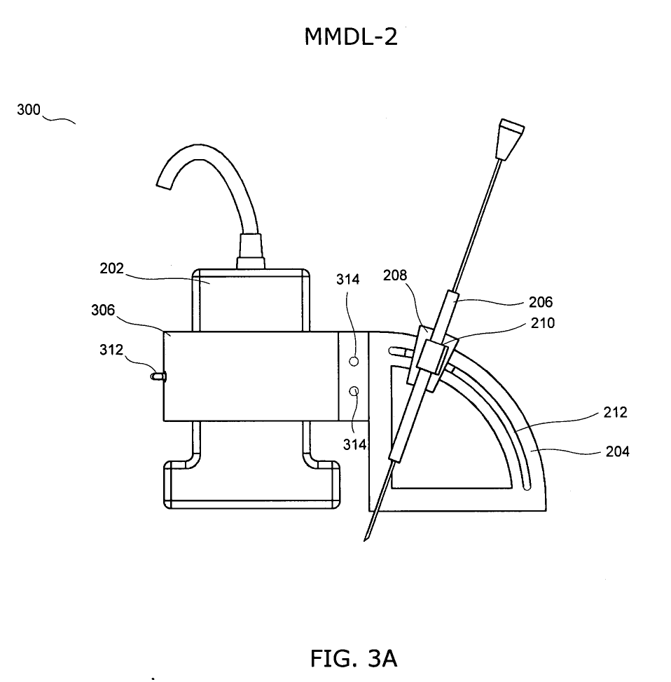

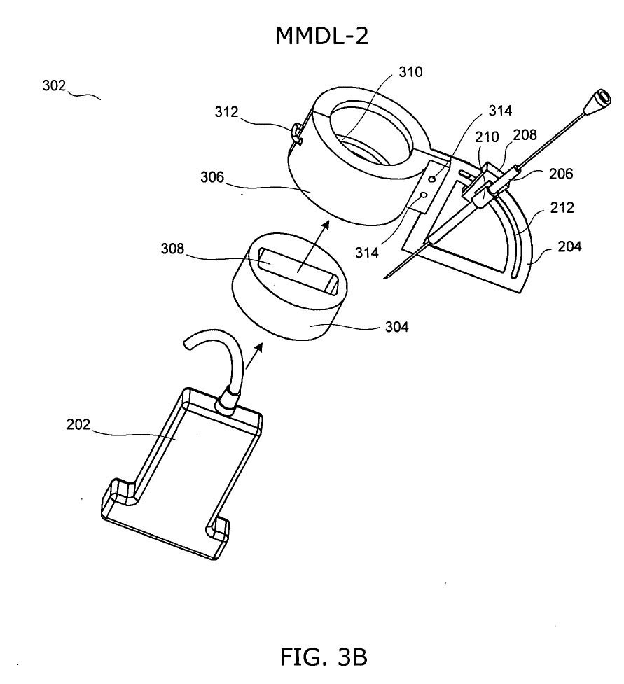

FIG. 3A illustrates a variable-angle needling device 300 that allows the medical imaging tool to rotate about an axis in accordance with a third example of the present disclosure, while FIG. 3B illustrates an isometric view 302 of how the variable-angle needling device 300 may be assembled in accordance with the third example of the present disclosure. With reference to FIGS. 3A and 3B, the variable-angle needling device 300 is similar to the variable-angle needling device 200 because it includes the protractor assembly 204, the arced slot 212 located in the protractor assembly 204, the clamp 210 into which the hollow guide tube 206 snaps, the bracket 208 which travels along the arced slot 212, as well as a position at which the ultrasonic transducer 202 may be adequately secured. The mechanics and functionalities of the aforementioned pieces are identical in the variable-angle needling devices 200 and 300.

With reference to FIGS. 2A, 3A and 3B, the variable-angle needling device 300 secures the ultrasonic transducer 202 not by a plurality of belts or straps 224 and 226, but through a rotating adapter 304 and a clamp ring 306. The rotating adapter 304 has a hollow 308 which allows the ultrasonic transducer 202 to fit tightly therein. The clamp ring 306 has a groove 310, in which the rotating adapter 304 fits and along which the rotating adapter 304 rotates. This rotational motion allows the ultrasonic transducer 202 to be introduced at various angles relative to the protractor assembly 204. It is understood that a different ultrasonic transducer may require a different rotating adapter, just as different electrical plugs may be needed for different electricity wall outlets. It is further understood that the clamp ring 306 may also include a plurality of engineering solutions, e.g. a lock screw, a belt-tightening mechanism etc. (not shown), that allow the ultrasonic transducer 202 to be fitted tightly into the hollow 308 of the rotating adapter 304. The clamp ring 306 may also have a lock mechanism 312, and may be attached to the protractor assembly 204, as an example, by using a plurality of screws 314.

This mechanism allows the ultrasonic transducer 202 to turn, through the rotating adapter 304, relative to the clamp ring 306, which is tightly attached to the protractor assembly 204. By providing such a rotational motion, the ultrasound transducer 202 may be introduced at a plurality of angles relative to the protractor assembly 204. While the aforesaid rotational motion is enabled through a rotating adapter and a clamp ring, it is understood that other engineering designs may exist, and that these designs may be implemented into the tool insertion guidance device to facilitate such a rotational motion without deviating from the spirit of the present disclosure.

The improved tool insertion guidance device, when used in conjunction with a medical imaging system, such as one comprising an ultrasonic transducer and its complementary tools, provides various advantages as shown in the previous examples. For example, the tool insertion guidance device assures that a surgical tool, such as a hypodermic needle, will always be inserted in the same plane as the imaging beam, and that the surgical tool is closely monitored as it travels to a targeted area. Since it is no longer necessary for the operator of the device to insert the needle by “trial-and-error”, the overall error rate, as well as the unnecessary pain suffered by the patients, will be significantly reduced. Further, by allowing the medical imaging tool, such as the ultrasonic transducer, of the medical imaging system to properly rotate relative to the tool insertion guidance device, more imaging information may be obtained. This additional information may further assist the operator of the device.

The above disclosure provides many different embodiments or examples for implementing different features of the disclosure. Specific examples of components and processes are described to help clarify the disclosure. These are, of course, merely examples and are not intended to limit the disclosure from that described in the claims.

Although the invention is illustrated and described herein as embodied in a design for guiding the insertion of a surgical tool, it is nevertheless not intended to be limited to the details shown, since various modifications and structural changes may be made therein without departing from the spirit of the invention and within the scope and range of equivalents of the claims. Accordingly, it is appropriate that the appended claims be construed broadly and in a manner consistent with the scope of the disclosure, as set forth in the following claims.

1. A tool insertion guidance device for guiding an insertion of a surgical tool to a target area monitored by a medical imaging tool, the device comprising:
a protractor assembly attached to the medical imaging tool having a predetermined slot;
a hollow guide tube held by the protractor assembly for receiving the surgical tool, the hollow guide tube assuring an insertion of the surgical tool to be within an imaging plane of the medical imaging tool; and
an adjusting means for moving a first end of the hollow guide tube along the slot for defining an insertion angle of the surgical tool.

2. The device of claim 1 further comprising a securing means for holding a second end of the hollow guide tube to the protractor assembly.

3. The device of claim 2 wherein the securing means is a set of securing pins.

4. The device of claim 1 wherein the adjusting means includes a set of guide pins for flexibly holding the first end of the hollow guide tube in the slot so that the first end of the tube is movable along the slot.

5. The device of claim 1 further comprising a locking means for locking the first end of the hollow guide tube to a predetermined location along the slot, thereby stabilizing the hollow guide tube.

6. The device of claim 5 wherein the locking means is a locking nut screwing on one of the guide pins.

7. The device of claim 1 wherein the protractor assembly has two protractor-like plates separated by a predetermined distance for placing the hollow guide tube therebetween.

8. The device of claim 1 wherein the protractor assembly has one protractor-like plate.

9. The device of claim 8 wherein the adjusting means is a bracket for attaching to the protractor assembly along the slot and with a clamp means for holding the hollow guide tube thereto.

10. The device of claim 9 wherein the clamp means is a snap-in clamp.

11. The device of claim 1 wherein the protractor assembly is of a quarter-circle shape and the slot is an arced slot.

12. The device of claim 1 wherein the surgical tool is a surgical needle.

13. The device of claim 1 wherein the medical imaging tool is an ultrasonic imaging tool.

14. The device of claim 1 wherein the slot has a teeth pattern track for stabilizing the hollow guide tube.

15. A guidance device for guiding an insertion of a needle to a target area monitored by an ultrasonic imaging tool, the device comprising:
a protractor assembly attached to the ultrasonic medical imaging tool;
a hollow guide tube held by the protractor assembly for receiving the needle, the hollow guide tube assuring an insertion of the needle to be within an imaging plane of the medical imaging system; and
an adjusting means for moving a first end of the hollow guide tube for defining an insertion angle of the needle.

16. The device of claim 15 wherein the protractor assembly has two protractor-like plates separated by a predetermined distance for placing the hollow guide tube therebetween.

17. The device of claim 16 further comprising a securing means for holding a second end of the hollow guide tube to the protractor assembly.

18. The device of claim 15 wherein the adjusting means includes a set of guide pins for flexibly holding the first end of the hollow guide tube in a moving track of the protractor assembly so that the first end of the tube is movable along the moving track.

19. The device of claim 18 further comprising a locking means for locking the first end of the hollow guide tube to a predetermined location along the moving track, thereby stabilizing the hollow guide tube.

20. The device of claim 15 wherein the protractor assembly has one protractor-like plate and the adjusting means is a bracket for attaching to the protractor assembly and with a clamp means for holding the hollow guide tube thereto.

21. The device of claim 20 wherein the clamp means is a snap-in clamp.

22. A guidance device for guiding an insertion of a needle to a target area monitored by an ultrasonic imaging tool, the device comprising:
a protractor assembly attached to the ultrasonic medical imaging tool with a moving track;
a hollow guide tube held by the protractor assembly for receiving the needle, the hollow guide tube assuring an insertion of the needle to be within an imaging plane of the medical imaging system;
an adjusting means for moving a first end of the hollow guide tube for defining an insertion angle of the needle; and
a locking means for locking the first end of the hollow guide tube to a predetermined location along the moving track, thereby stabilizing the hollow guide tube.

23. The device of claim 22 wherein the adjusting means includes a set of guide pins for flexibly holding the first end of the hollow guide tube in the slot so that the first end of the tube is movable along the moving track.

24. The device of claim 22 wherein the adjusting means is a bracket for attaching to the protractor assembly along the moving track and with a clamp means for holding the hollow guide tube thereto.

25. The device of claim 22 wherein the protractor assembly is of a quarter-circle shape and the moving track is an arced slot.
